(19) United States
(12) Patent Application Publication
(10) Pub. No.: US 2026/0134949 A1
(43) Pub. Date: May 14, 2026

(54) ENCODED DNA DATA STORAGE METHOD WITH ACCOMPANYING COMPOSITE CODES AS INDEX

(71) Applicant: TIANJIN UNIVERSITY, Tianjin (CN)

(72) Inventors: Weigang CHEN, Tianjin (CN); Yuxin ZHANG, Tianjin (CN); Changcai HAN, Tianjin (CN); Rui QIN, Tianjin (CN)

(21) Appl. No.: 19/380,016

(22) Filed: Nov. 5, 2025

(30) Foreign Application Priority Data
Nov. 8, 2024 (CN) 202411594023.X

(51) Int. Cl. G16B 50/30; C12N 15/10

(52) U.S. Cl. CPC G16B 50/30; C12N 15/1065

(57) ABSTRACT

The present invention discloses an encoded DNA data storage method with accompanying composite codes as index, including using a composite code constructed from a limited number of pseudo-random component codes as an index sequence, performing accompanying encoding and mapping with an error-correction-coded data bit sequence, and combining to obtain a large DNA fragment or a short oligonucleotide molecule; and during readout, randomly fragmented large DNA or an oligonucleotide pool are read out via next-generation high-throughput sequencing technology, and demapping into two layers of bit sequences, then performing sliding correlation acquisition between corrupted composite code and known pseudo-random component codes with a relatively small number of correlations, determining a unique index according to calculation with the Chinese Remainder Theorem, and finally, performing consensus and decoding on a corrupted data sequence.

### CROSS-REFERENCE TO RELATED APPLICATION

This application claims priority from the Chinese patent application 202411594023.X filed Nov. 8, 2024, the content of which is incorporated herein in the entirety by reference.

### TECHNICAL FIELD

The present invention relates to the field of DNA storage technology, and in particular, to an encoded DNA data storage method with accompanying composite codes as index.

### BACKGROUND

The rapid development of information technology has accelerated the generation of data. According to a forecast by International Data Corporation (IDC), the total volume of global data will reach 175 ZB by 2025. As we enter the era of the digital economy, data, as a new production factor, plays an increasingly important role in social production activities. According to the Decadal Plan for Semiconductors released by the Semiconductor Industry Association (SIA) and Semiconductor Research Corporation (SRC) in 2021, more than 60% of all stored data will become cold data that does not require frequent access. According to a report released by Horizons, data has the characteristic that its value may become important again as time passes. With the development of big data-based artificial intelligence technology, the value of long-term data storage has become increasingly prominent. Long-term data storage has created a demand for long-term storage of massive data. However, existing data storage methods based on magnetic, optical, and electrical technologies generally have a service life of no more than several decades, with high maintenance costs and difficulties in increasing density, which cannot meet the needs of explosively growing data storage.

DNA data storage uses deoxyribonucleic acid (DNA) as a novel data storage medium. It boasts core advantages such as high storage density, long service life, and low maintenance costs, making it a highly promising new mode for the long-term and stable storage of massive data. In particular, it has broad application prospects in the long-term storage of archived data. According to the Semiconductor Synthetic Biology Roadmap released by the National Institute of Standards and Technology (NIST) and SRC, compared with high-capacity magnetic tapes with the highest storage density, DNA data storage methods offer approximately 7-order-of-magnitude improvement in storage density. According to a forecast by the Intelligence Advanced Research Projects Activity (IARPA), adopting DNA storage in EB-scale data centers is expected to reduce power consumption from 200 MW to 200 KW. Meanwhile, DNA data storage can achieve offline storage, featuring high security and ease of backup. It is expected to become a potential solution for the long-term and stable storage of massive archived data in the future. DNA data storage is a highly cutting-edge emerging technology. In 2023, the UK Research and Innovation listed DNA data storage as one of the 50 most promising emerging technologies for the future, and it is among the 8 technologies in the direction of “Artificial Intelligence, Digital, and Computing Technologies.” In 2023, publications such as the International Roadmap for Devices and Systems released by the Institute of Electrical and Electronics Engineers (IEEE) have identified DNA storage as one of the primary storage media for massive data in the future.

In recent years, a series of breakthrough advances have been made in proof-of-concept research on DNA data storage. Currently, mainstream DNA data storage modes mainly include DNA storage with short fragments and DNA storage with large fragments. For data storage with short fragments, an oligonucleotide pool composed of a large number of short-strand DNA molecules (typically 100-300 bases) is utilized to store data, and typically employs next-generation high-throughput sequencing for readout. For data storage with large fragments, data readout can be achieved using technologies similar to genome sequencing, including next-generation high-throughput sequencing and third-generation nanopore sequencing.

Regardless of the mode, DNA data storage distributes information across a large number of short DNA fragments, and restoring the correct order of all fragments is key to the reliable recovery of data. Therefore, how to achieve efficient indexing and rapid readout of data stored in DNA remains a critical technical challenge that urgently needs to be solved in DNA data storage.

For data storage with large fragments, if next-generation high-throughput sequencing is used for readout, similar to genome sequencing, the enriched DNA is typically randomly fragmented into a large number of short fragments via shotgun sequencing, and the reconstruction of the original data sequence is achieved through a de novo assembly strategy. However, the fragmentation positions of shotgun sequencing and the lengths of the resulting short DNA fragments are random, leading to difficulties in indexing, which requires the use of complex graph-based methods relying on overlap. To improve the accurate positioning of contigs during de novo assembly and avoid gaps as much as possible, the sequencing coverage required for data recovery is typically relatively high. Currently, software for assembling short fragments from next-generation sequencing is mainly based on the de Bruijn graph algorithm, and high sequencing coverage leads to an increase in the number of k-mer nodes during graph construction, further increasing computational complexity. Researchers have employed de Bruijn graph-based assembly software tools, e.g., Velvet and AbySS, to perform de novo assembly of next-generation sequencing reads. With the assistance of pilot sequences, they have recovered payload data using sequence alignment algorithms. The sequencing coverage required for achieving error-free recovery of data using both assembly software tools exceeds 20×. Researchers have employed the minimum sequencing coverage required for assembly (typically at least no less than 20×) to perform de novo assembly recovery of data-carrying large DNA fragments. Researchers have employed the graph-based short-read assembly software, SOAPdenovo2, for de novo assembly, combined with contig alignment and consensus to reconstruct data sequences, but the processing complexity is extremely high.

For data storage with short fragments, i.e., data storage with regularly segmented short DNA oligonucleotides, a large number of oligonucleotide molecules exhibit unordered characteristics, and the order of readout is random. Therefore, it is generally necessary to attach an additional short base sequence as a unique index to each oligonucleotide molecule to identify the position of the data carried by the molecule within the entire dataset. Meanwhile, during actual DNA storage and reading processes, DNA molecules are at risk of degradation, leading to a decline in quality, and are highly susceptible to hydrolysis in aqueous solutions, resulting in frequent DNA strand breaks, which thus pose a threat to the reliability of DNA data storage. To address the issue of DNA strand breaks, researchers have proposed sequence reconstruction and error correction algorithms based on de Bruijn graph theory, and have validated the high robustness of data recovery through accelerated aging experiments involving sample breakage and degradation. However, sequence reconstruction algorithms based on de Bruijn graphs generally rely on high sequencing coverage and have high computational complexity, making them difficult to meet the demand for rapid data readout.

### SUMMARY

To overcome the shortcomings of existing technologies, the present invention provides an encoded DNA data storage method with accompanying composite codes as index. The present invention enables rapid and robust identification of large-scale short data-carrying DNA at arbitrary positions with extremely low complexity, tolerates partial loss within fragments, and is applicable to both short-fragment and long-fragment storage modes, as described in detail below:

An encoded DNA data storage method with accompanying composite codes as index, including the following steps:

(1) at a data writing end, selecting n pseudo-random sequences as component codes, where n is a positive integer, and constructing a composite code as an index through combination logic, encoding user data using error correction codes to obtain an encoded data bit sequence, then performing accompanying encoding with bitwise combination on the composite code and the data bit sequence to form a dual-layer bit sequence, and transcoding it to obtain a data DNA sequence according to a mapping rule between a bit pair and a base, and finally completing data writing through artificial DNA synthesis; and

(2) at a data reading end, obtaining sequencing reads of a short-fragment DNA using next-generation high-throughput sequencing technology, preprocessing the sequencing reads, performing demapping according to the same mapping rule as the writing end, extracting a corrupted composite code and a corrupted data sequence, next, performing sliding correlation detection with the corrupted composite code using n known component codes respectively to obtain a correlation peak phase estimate for each component code, then performing calculation through the Chinese Remainder Theorem to determine unique indices of reads, then calculating an in-phase autocorrelation value of the composite code and performing threshold check, retaining reads that pass the check as they are while re-identifying reads that fail the check using an enhanced mode, and locating and correcting an insertion/deletion error in the corrupted data sequence, and finally, according to identified indices, using a plurality of copies of a sequence at the same position, performing a bitwise majority voting to generate a consensus data sequence, and sending it to a decoder for decoding to recover original user data.

The step (1) is specifically as follows:
(1.1) selecting n pseudo-random sequences as component codes to form a component code set {C₁, C₂, . . . , Cₙ}, then subjecting each component code to periodic extension to obtain sequences each with a length of P=p₁p₂ . . . pₙ, and performing bitwise calculation according to preset Boolean combination logic to construct a composite code a with a length of P=p₁p₂ . . . pₙ, and representing the composite code in binary form; where Cₙ denotes a component code n with a code length of pₙ, and n≥1 and is an integer;
(1.2) performing block error correction encoding on user data using m linear block codes (N,K) to obtain an encoded data bit sequence b; where m is the number of code words, m≥1 and is an integer, N is a length of an encoded code word, and K is a length of information bits; and since a period of the composite code can be designed to be extremely long, a single composite code can carry a plurality of block codewords, satisfying mN≤p₁p₂ . . . pₙ; and
(1.3) performing accompanying encoding with bitwise combination on the composite code a and the data bit sequence b to construct a dual-layer bit sequence, transcoding it into a DNA sequence according to a mapping rule between a bit pair and a base {00→A, 01→T, 10→G, 11→C}, and obtaining a long DNA fragment or a short oligonucleotide molecule by means of artificial synthesis technology;
where the preset Boolean combination logic specifically includes: logical multiplication, modulo-2 addition, and majority decision;
where the n pseudo-random sequences with short periods are selected as the component codes, satisfying: periods of the selected pseudo-random sequences are strictly coprime to each other, and each has sharp periodic autocorrelation characteristics; and
where the step (1.3) is specifically as follows:
for data storage with large fragments, first intercepting the composite code to obtain a shortened composite code with the same length as the data bit sequence, then aligning the shortened composite code and the data bit sequence end-to-end to form a dual-layer bit sequence, and directly performing mapping to obtain long DNA fragments; and for data storage with short oligonucleotides, first dividing the data bit sequence evenly into mN/l groups according to a length l, simultaneously performing sliding interception on the composite code according to a step size 1 to obtain mN/l shortened composite codes, then pairing the shortened composite codes with a length l and the data bit sequence in order to construct mN/l groups of dual-layer bit sequences, and finally, generating mN/l short-fragment oligonucleotides after mapping; where l is a positive integer.

The step (2) is specifically as follows:
(2.1) for a short-fragment oligonucleotide molecule pool, after direct library preparation, reading through next-generation high-throughput sequencing technology to obtain sequencing reads of a data DNA; and for a long-fragment data DNA, performing sequencing readout using high-throughput shotgun sequencing, and specifically, randomly fragmenting enriched DNA molecules, and reading through next-generation high-throughput sequencing to obtain sequencing reads;
(2.2) preprocessing the sequencing reads, performing demapping according to the same mapping rules as the writing end, and separately extracting a corrupted composite code â and a corrupted data bit sequence \hat{b} from a dual-layer structure; specifically, for sequencing reads of a short-fragment oligonucleotide, trimming off primer regions at both ends by aligning with amplification primers, and performing demapping on a retained payload; and for reads obtained by shotgun sequencing of a long-fragment DNA, performing demapping directly on the sequencing reads;
(2.3) sending the corrupted composite code â into a cross-correlation detector of each component code, performing sliding correlation acquisition using a known component code set {C₁, C₂, . . . , Cₙ}, searching for a position of a correlation peak of each component code (i.e., a phase corresponding to in-phase correlation), obtaining an estimated phase of each component code, and then calculating through the Chinese Remainder Theorem to determine unique indices of reads;
(2.4) regenerate an ideal composite code a′ with the same length and phase as the corrupted composite code using the above obtained estimated phase of each component code, calculating the autocorrelation value of the composite code, and if an autocorrelation value meets a preset condition, retaining reads; and for reads that fail a check, performing re-identification using an edit distance alignment-based method in an enhanced mode to determine indices, and retaining the reads, while identifying and correcting insertion and deletion error patterns according to an alignment path; and
(2.5) considering that a plurality of copies of a data sequence may exist at the same position, performing a bitwise majority voting decision on the data sequence to obtain a consensus data sequence, and if no uniqueness exists in a voting decision, filling a bit at the position using the same erasure marker as above to obtain a codeword, and then sending it to a decoder to correct residual errors and recovering original user data.

The step (2.3) is specifically as follows:
(3.1) subjecting each component code in a known component code set to periodic extension, subjecting each component code to sliding correlation operation with the corrupted composite code, where a correlation window length is the same as a length of the corrupted composite code, a sliding number is a component code period, and a cross-correlation function between a component code i and the corrupted composite code can be expressed as:

$${R}_{\overline{r} , {\overline{C}}_{i}} ( τ ) = {\sum }_{j = 0}^{L - 1} \overline{r} ( j ) {\overline{C}}_{i} ( j - τ ) ,$$

where C̅₁ is a periodic extension sequence of the component code i, r̅ is the corrupted composite code, τ is a component code phase offset, L is the correlation window length, 1≤i≤n, the periodic extension sequence C̅ᵢ and the composite code r̅ are represented by symbols {+1, −1}, which satisfy a mapping relationship {+1→0, −1→1} with a binary sequence;
(3.2) according to a sliding correlation value between each component code and the corrupted composite code, searching for a position of a correlation peak to obtain a phase estimate of each component code, where a phase τᵢ corresponding to the correlation peak of the component code i can be expressed as:

$${τ}_{i} = {arg max}_{τ} ( \vert {R}_{\overline{r} , {\overline{C}}_{1}} ( τ ) \vert ) ,$$

where argmax represents obtaining the position corresponding to the correlation peak; and
(3.3) using an estimated phase of each component code obtained from the searching, calculating through the Chinese Remainder Theorem to obtain a unique index of a corresponding read, where the estimated phase of each component code satisfies

$$\{ \begin{matrix}φ ≡ {τ}_{1} ( mod {p}_{1} ) \\ φ ≡ {τ}_{2} ( mod {p}_{2} ) \\ ⋮ \\ ϕ ≡ {τ}_{n} ( mod {p}_{n} )\end{matrix} ,$$

where φ is the unique index of the read obtained through calculation, and τ₁, τ₂, . . . , and τₙ correspond to estimated phases of n component codes, respectively.

The step (2.4) is specifically as follows:
(4.1) according to the estimated phase of each component code mentioned above, regenerating an ideal composite code a′ with the same phase and length as the corrupted composite code â, and then calculating an in-phase correlation value between the corrupted composite code â and the ideal composite code a′, where the obtained correlation value can be expressed as:

$${R}_{r , {r}^{′}} = {\sum }_{j = 0}^{L - 1} r ( j ) {r}^{′} ( j ) ,$$

where r and r′ are the corrupted composite code and the ideal composite code, respectively, both represented by symbols {+1, −1}, and satisfy a mapping relationship {+1→0, −1→1} with the binary sequence;
(4.2) if the in-phase autocorrelation value of the composite code satisfies |R_{r,r′}|≥ωL, passing a check, and retaining a current read, where w represents a preset correlation threshold, and 0≤ω≤1;
(4.3) for reads that fail the above autocorrelation check, in an enhanced mode, adopting an edit distance alignment-based method to compare the corrupted composite code â with the known complete composite code a, searching for an alignment position with a minimum edit distance as an index of a read, while locating positions with insertion and deletion errors in the corrupted composite code using an alignment path, and according to a characteristic that positions with insertion and deletion errors in a dual-layer sequence structure are completely identical, correcting insertion and deletion error patterns in the corrupted data sequence; discarding bits at positions with insertion errors, marking bits at positions with deletion errors as erasures and filling them with a predefined marker; and if the enhanced mode is not adopted, skipping this step.

A second aspect of the present invention further includes a system for implementing encoded DNA data storage with accompanying composite codes as index, including: a data writing module and a data reading module;
where the data writing module includes an encoder and a high-throughput synthesizer; the encoder is configured to select n pseudo-random sequences as component codes, where n is a positive integer, and construct a composite code as an index through combination logic, encode user data using error correction codes to obtain an encoded data bit sequence, then perform accompanying encoding with bitwise combination on the composite code and the data bit sequence to form a dual-layer bit sequence, and transcode it to obtain a data DNA sequence according to a mapping rule between a bit pair and a base;
the high-throughput synthesizer is configured for highly parallel DNA synthesis to complete data writing and obtain a large-scale oligonucleotide pool carrying information;
the data reading module includes a high-throughput sequencer and a decoder; the high-throughput sequencer is configured to perform highly parallel sequencing on a DNA library sample prepared by library preparation, and generate sequencing reads through base calling; and
the decoder is configured to preprocess the sequencing reads, perform demapping according to the same mapping rule as a writing end, extract a corrupted composite code and a corrupted data sequence, next, perform sliding correlation detection with the corrupted composite code using known component codes respectively to obtain a correlation peak phase estimate for each component code, then perform calculation through the Chinese Remainder Theorem to determine unique indices of reads, then calculate an in-phase autocorrelation value of the composite code and perform threshold check, retain reads that pass the check as they are while re-identify reads that fail the check using an enhanced mode, and locate and correct an insertion/deletion error in the corrupted data sequence, and finally, according to identified indices, using multiple copies of a sequence at the same position, perform a bitwise majority voting to generate a consensus data sequence, and send it to a decoder for decoding to recover original user data.

Compared with the prior art, the beneficial effects of the technical solutions provided by the present invention are as follows:

(1) The present invention makes full use of the single-period ambiguity-free characteristic of the composite code and the good cross-correlation characteristics between the component codes and the composite code. The composite code generated by a limited number of short-period component codes is used as the accompanying index sequence, and is combined with the original data sequence to construct a DNA sequence. During data readout, sliding correlation detection with a limited length of component code periods is performed, and then the actual position of the read is calculated using the Chinese Remainder Theorem, thereby quickly identifying the unique index of the sequencing read and significantly improving the indexing efficiency of readout and recovery in large-scale data storage.

(2) The present invention is applicable to both storage modes using large DNA fragments and short oligonucleotide pools, enabling robust identification of DNA fragments with arbitrary position, and avoiding the complex assembly operations required for sequence reconstruction in large-fragment shotgun sequencing. For oligonucleotide pools with fixed starting positions, partial loss within fragments is allowed, thereby improving the readout efficiency of DNA oligonucleotide pools.

### BRIEF DESCRIPTION OF THE DRAWINGS

FIG. 1 is a block diagram of system implementation of an encoded DNA data storage method with accompanying composite codes as index provided by the present invention;

FIG. 2 is a flow chart of implementation of encoding with accompanying composite code for constructing storage modes with large DNA fragments or short oligonucleotide pools in the present invention;

FIG. 3 is a flow block diagram of read index identification and data recovery for next-generation sequencing readout provided by the present invention;

FIG. 4 is a flow chart of composite code construction provided by the present invention;

FIG. 5 is a flow chart of implementation of rapid read identification based on short-period component code sliding correlation detection provided by the present invention;

FIG. 6 is a performance comparison graph of a read identification success rate and a usable read ratio under different normalized thresholds in Example 1 of the present invention;

FIG. 7 is a performance graph of a fragment identification success rate at different normalized autocorrelation thresholds after adopting an enhanced mode in Example 1 of the present invention;

FIG. 8 is a performance graph of an error rate after multi-copy consensus under different sequencing coverages in Example 1 of the present invention;

FIG. 9 is a comparison graph of time complexity in Example 1 of the present invention;

FIG. 10 is a performance comparison graph of a read identification success rate and a usable read ratio under different normalized thresholds in Example 2 of the present invention;

FIG. 11 is a performance graph of an error rate after multi-copy consensus under different sequencing coverages in Example 2 of the present invention;

FIG. 12 shows error-free recovery of 50 repeated experiments under different sequencing coverages in Example 3 of the present invention; and

FIG. 13 is a structural diagram of an encoded DNA data storage system with accompanying composite codes as index in Example 4.

### DESCRIPTION OF THE EMBODIMENTS

To make the objectives, technical solutions, and advantages of the present invention clearer, the embodiments of the present invention are described in further detail below.

Aiming at the problem of low efficiency of sequence indexing in DNA application scenarios with short fragments and large fragments, the present invention provides a new encoding method universally applicable to both DNA data storage with short fragments and large fragments, i.e. an encoded DNA data storage method with accompanying composite codes as index. This method constructs a DNA sequence by combining a composite code segment formed by logically combining a limited number of pseudo-random sequences with a data segment. During readout, it enables fast and efficient indexing of a DNA sequencing read with an arbitrary start sites (shotgun large-fragment sequencing mode) and a fixed start sites (oligonucleotide pool sequencing readout mode), thereby achieving rapid data recovery. The method can handle partial loss in a fixed-segment short-fragment DNA sequence, thus improving readout efficiency.

The examples of the present invention are described in detail below in conjunction with the drawings:

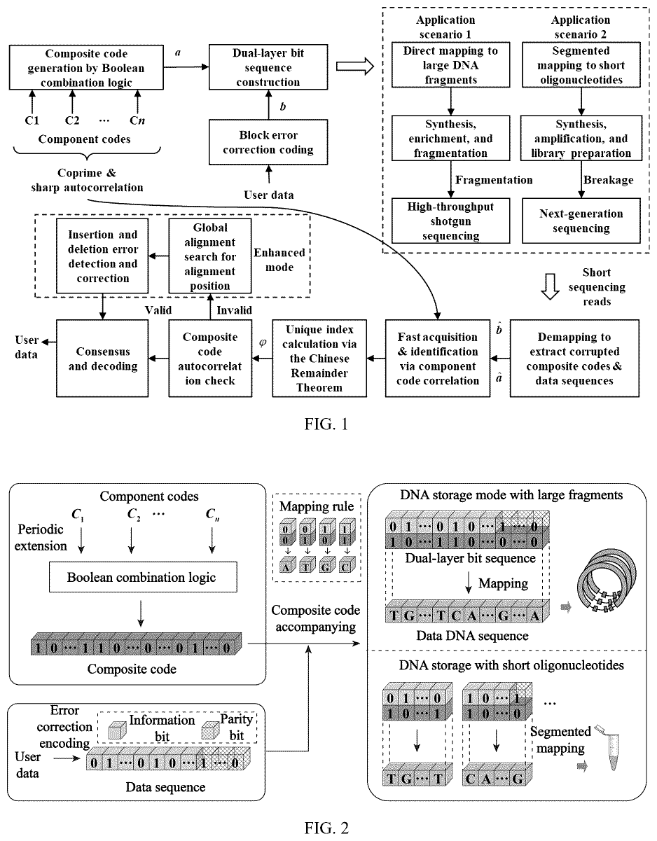

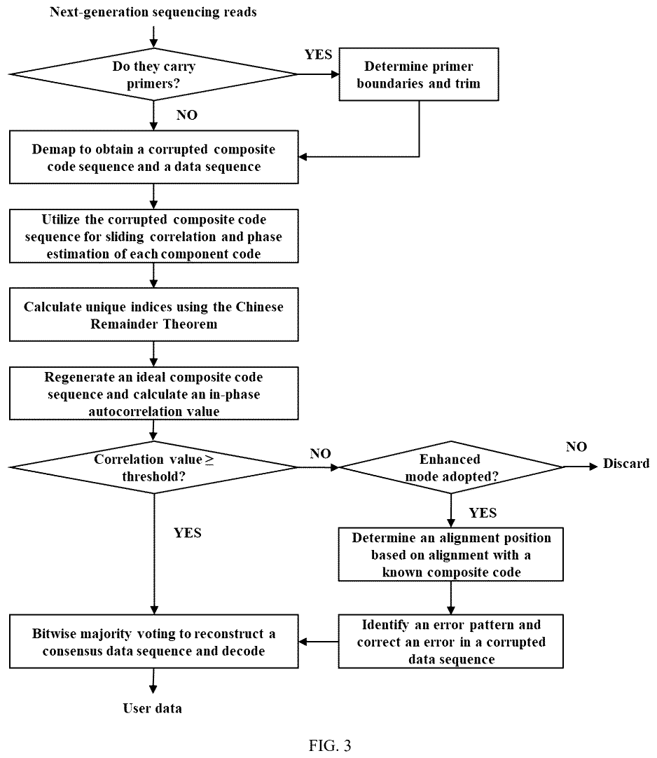

This example provides a detailed introduction to the encoded DNA data storage method with accompanying composite codes as index proposed by the present invention. The block diagram of overall system implementation is shown in FIG. 1, which specifically includes the following steps:
(1) at a data writing end, see FIG. 2, selecting n pseudo-random sequences as component codes, where n is a positive integer, and constructing a composite code as an index through combination logic, encoding user data using error correction codes to obtain an encoded data bit sequence, then performing accompanying encoding with bitwise combination on the composite code and the data bit sequence to form a dual-layer bit sequence, and transcoding it to obtain a data DNA sequence according to a mapping rule between a bit pair and a base, and finally completing data writing through artificial DNA synthesis; and
(2) at a data reading end, see FIG. 3, obtaining sequencing reads of a short-fragment DNA using next-generation high-throughput sequencing technology, preprocessing the sequencing reads, performing demapping according to the same mapping rule as the writing end, extracting a corrupted composite code and a corrupted data sequence, next, performing sliding correlation detection with the corrupted composite code using n known component codes respectively to obtain a correlation peak phase estimate for each component code, then performing calculation through the Chinese Remainder Theorem to determine unique indices of reads, then calculating an in-phase autocorrelation value of the composite code and performing threshold check, retaining reads that pass the check as they are while re-identifying reads that fail the check using an enhanced mode, and locating and correcting an insertion/deletion error in the corrupted data sequence, and finally, according to identified indices, using a plurality of copies of a sequence at the same position, performing a bitwise majority voting to generate a consensus data sequence, and sending it to a decoder for decoding to recover original user data.

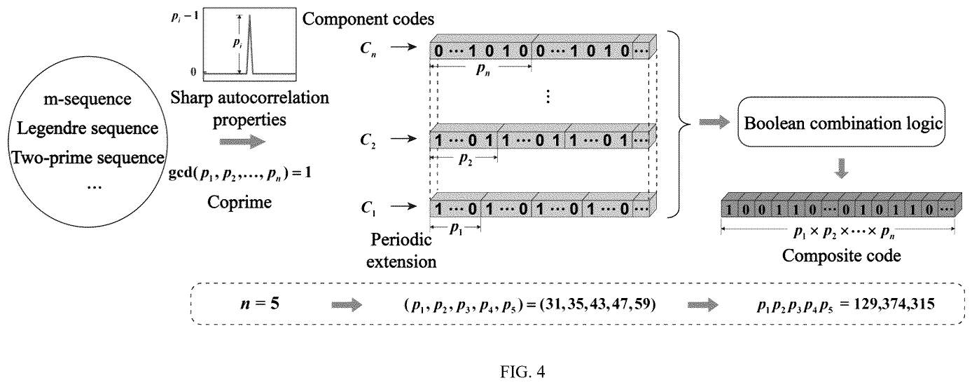

The step (1) is specifically as follows:
(1.1) as shown in FIG. 4, selecting n pseudo-random sequences as component codes to form a component code set {C₁, C₂, . . . , Cₙ}, then subjecting each component code to periodic extension to obtain sequences each with a length of P=p₁p₂ . . . pₙ, and performing bitwise calculation according to preset Boolean combination logic to construct a composite code a with a length of P=p₁p₂ . . . pₙ, and representing the composite code in binary form; where Cₙ denotes a component code n with a code length of pₙ, and n≥1 and is an integer;
(1.2) performing block error correction encoding on user data using m linear block codes (N,K) to obtain an encoded data bit sequence b; where m is the number of codewords, m≥1 and is an integer, N is a length of an encoded codeword, and K is a length of information bits; and since a period of the composite code can be designed to be extremely long, a single composite code can carry a plurality of block codewords, satisfying mN≤p₁p₂ . . . pₙ; and
(1.3) performing accompanying encoding with bitwise combination on the composite code a and the data bit sequence b to construct a dual-layer bit sequence, transcoding it into a DNA sequence according to a mapping rule between a bit pair and a base {00→A, 01→T, 10→G, 11→C}, and obtaining a long DNA fragment or a short oligonucleotide molecule by means of artificial synthesis technology;
where the preset Boolean combination logic specifically includes: logical multiplication, modulo-2 addition, majority decision, and the like;
where the n pseudo-random sequences with short periods are selected as the component codes, satisfying: periods of the selected pseudo-random sequences are strictly coprime to each other, and each has sharp periodic autocorrelation characteristics; and
where the step (1.3) is specifically as follows:
for data storage with large fragments, first intercepting the composite code to obtain a shortened composite code with the same length as the data bit sequence, then aligning the shortened composite code and the data bit sequence end-to-end to form a dual-layer bit sequence, and directly performing mapping to obtain long DNA fragments; and for data storage with short oligonucleotides, first dividing the data bit sequence evenly into mN/l groups according to a length l, simultaneously performing sliding interception on the composite code according to a step size l to obtain mN/l shortened composite codes, then pairing the shortened composite codes with a length l and the data bit sequence in order to construct mN/l groups of dual-layer bit sequences, and finally, generating mN/l short-fragment oligonucleotides after mapping; where l is a positive integer.

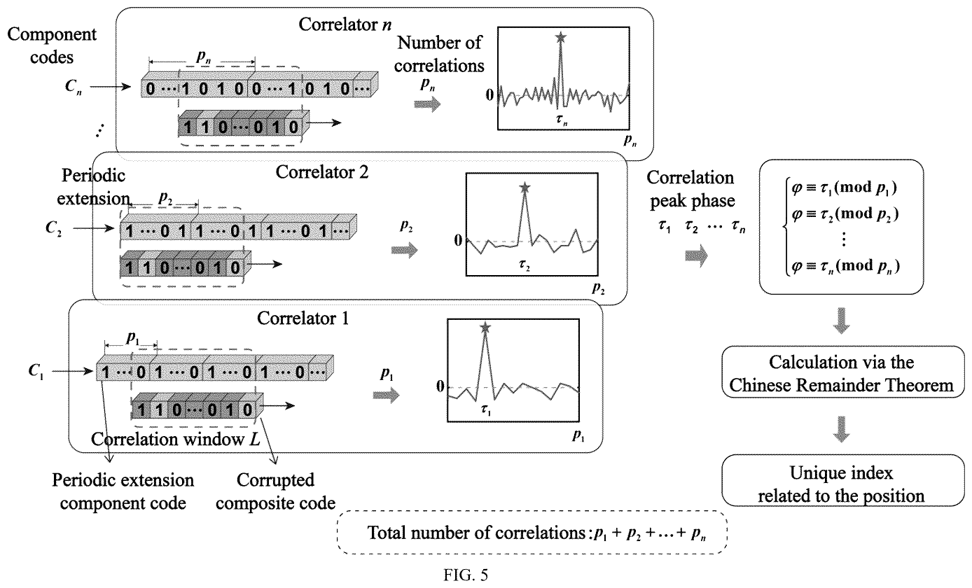

The step (2) is specifically as follows:
(2.1) for a short-fragment oligonucleotide molecule pool, after direct library preparation, reading through next-generation high-throughput sequencing technology to obtain sequencing reads of a data DNA; and for a long-fragment data DNA, performing sequencing readout using high-throughput shotgun sequencing technology, and specifically, randomly fragmenting enriched DNA molecules, and reading through next-generation high-throughput sequencing technology to obtain sequencing reads;
(2.2) preprocessing the sequencing reads, performing demapping according to the same mapping rules as the writing end, and separately extracting a corrupted composite code â and a corrupted data bit sequence \hat{b} from a dual-layer structure; specifically, for sequencing reads of a short-fragment oligonucleotide, trimming off primer region sat both ends by aligning with amplification primers, and performing demapping on a retained payload; and for reads obtained by shotgun sequencing of a long-fragment DNA, performing demapping directly on the sequencing reads;
(2.3) as shown in FIG. 5, sending the corrupted composite code â into a cross-correlation detector of each component code, performing sliding correlation acquisition using a known component code set {C₁, C₂, . . . , Cₙ} searching for a position of a correlation peak of each component code (i.e., a phase corresponding to in-phase correlation), obtaining an estimated phase of each component code, and then calculating through the Chinese Remainder Theorem to determine unique indices of reads;
(2.4) regenerate an ideal composite code a′ with the same length and phase as the corrupted composite code using the above obtained estimated phase of each component code, calculating the autocorrelation value of the composite code, and if an autocorrelation value meets a preset condition, retaining reads; and for reads that fail a check, performing re-identification using an edit distance alignment-based method in an enhanced mode to determine indices, and retaining the reads, while identifying and correcting insertion and deletion error patterns according to an alignment path; and
(2.5) considering that multiple copies of a data sequence may exist at the same position, performing a bitwise majority voting decision on the data sequence to obtain a consensus data sequence, and if no uniqueness exists in a voting decision, filling a bit at the position using the same erasure marker as above to obtain a codeword, and then sending it to a decoder to correct residual errors and recovering original user data.

The step (2.3) is specifically as follows:
(3.1) subjecting each component code in a known component code set to periodic extension, subjecting each component code to sliding correlation operation with the corrupted composite code, where a correlation window length is the same as a length of the corrupted composite code, a sliding number is a component code period, and a cross-correlation function between a component code i and the corrupted composite code can be expressed as:

$${R}_{\overline{r} , \overline{{C}_{i}}} ( τ ) = {\sum }_{j = 0}^{L - 1} \overline{r} ( j ) {\overline{C}}_{i} ( j - τ ) ,$$

where C̅ᵢ is a periodic extension sequence of the component code i, r̅ is the corrupted composite code, τ is a component code phase offset, L is the correlation window length, 1≤i≤n, the periodic extension sequence C̅ᵢ and the composite code r̅ are represented by symbols {+1, −1}, which satisfy a mapping relationship {+1→0, −1→1} with a binary sequence;
(3.2) according to a sliding correlation value between each component code and the corrupted composite code, searching for a position of a correlation peak to obtain a phase estimate of each component code, where a phase τᵢ corresponding to the correlation peak of the component code i can be expressed as:

$${τ}_{i} = {arg max}_{τ} ( \vert {R}_{\overline{r} , {\overline{C}}_{i}} ( τ ) \vert ) ,$$

where argmax represents obtaining the position corresponding to the correlation peak; and
(3.3) using an estimated phase of each component code obtained from the searching, calculating through the Chinese Remainder Theorem to obtain a unique index of a corresponding read, where the estimated phase of each component code satisfies:

$$\{ \begin{matrix}φ ≡ {τ}_{1} ( mod {p}_{1} ) \\ φ ≡ {τ}_{2} ( mod {p}_{2} ) \\ ⋮ \\ φ ≡ {τ}_{n} ( mod {p}_{n} )\end{matrix} ,$$

where φ is the unique index of the read obtained through calculation, and τ₁, τ₂, . . . , and τₙ correspond to estimated phases of n component codes, respectively.

The step (2.4) is specifically as follows:
(4.1) according to the estimated phase of each component code mentioned above, regenerating an ideal composite code a′ with the same phase and length as the corrupted composite code â, and then calculating an in-phase correlation value between the corrupted composite code â and the ideal composite code a′, where the obtained correlation value can be expressed as:

$${R}_{\overline{r} , {r}^{′}} = {\sum }_{j - 0}^{L - 1} \overline{r} ( j ) {r}^{′} ( j ) ,$$

where r̅ and r′ are the corrupted composite code and the ideal composite code, respectively, both represented by symbols {+1, −1}, and satisfy a mapping relationship {+1→0, −1→1} with the binary sequence;
(4.2) if the in-phase autocorrelation value of the composite code satisfies |R_{r̅,r′}|≥ωL, passing a check, and retaining a current read, where ω represents a preset correlation threshold, and 0≤ω≤1;
(4.3) for reads that fail the above autocorrelation check, in an enhanced mode, adopting an edit distance alignment-based method to compare the corrupted composite code â with the known complete composite code a, searching for an alignment position with a minimum edit distance as an index of a read, while locating positions with insertion and deletion errors in the corrupted composite code using an alignment path, and according to a characteristic that positions with insertion and deletion errors in a dual-layer sequence structure are completely identical, correcting insertion and deletion error patterns in the corrupted data sequence; specifically, discarding bits at positions with insertion errors, marking bits at positions with deletion errors as erasures and filling them with a predefined marker; and if the enhanced mode is not adopted, skipping this step.

According to the encoded DNA data storage method with accompanying composite codes as index proposed by the present invention, by utilizing the good correlation characteristics of the composite code and the distinguishable property of arbitrary phase within a single period, the composite code constructed from a limited number of short-period pseudo-random component codes is used as the index, and combined with the data sequence to form a dual-layer sequence structure. After mapping, the DNA sequence is obtained. During data readout, combined with the Chinese Remainder Theorem, rapid read identification can be achieved only by performing sliding correlation detection of component code periods using a limited number of component codes. This method supports robust identification of DNA fragments with arbitrary start sites and can tolerate partial loss within fragments, making it universally applicable to both storage modes with large DNA fragments and short oligonucleotides via next-generation sequencing readout.

Two specific examples are provided below, and the feasibility of the present invention is illustrated in conjunction with the drawings to further understand the objectives, features, and advantages of the present invention. In the four specific examples provided, the Boolean combination logic for constructing the composite code adopts majority logic, and its expression can be expressed as ā=sign(C̅₁+C̅₂+ . . . +C̅ₙ) where C̅ₙ represents a symbol sequence corresponding to a component code n, which consists of {+1, −1} and satisfies a mapping relationship with a bit sequence {−1→1, +1→0}, and the number of component codes n satisfies n>1 and is an odd number.

### Example 1

In this example, a composite code constructed from 5 component codes was adopted as the index of a data DNA. The identification performance of 130-nucleotide sequencing reads was simulated to verify the low complexity and high robustness of the indexing method proposed by the present invention. A component code set was formed by selecting 5 pseudo-random sequences with periods of 31, 35, 43, 47, and 59, respectively, where the component code with a period of 31 is an m-sequence, the component code with a period of 35 is a two-prime sequence, and the component codes with periods of 43, 47, and 59 are Legendre sequences. Table 1 presents the bit sequence corresponding to each component code.

TABLE 1
5 pseudo-random component codes used to construct the composite code

| Component | Code | |
|---|---|---|
| code | length | Bit sequence |
| C₁ | 31 | 1111011010011000001110010001010 |
| C₂ | 35 | 11011001010111101100010000011100010 |
| C₃ | 43 | 1011010110001000001110100011111011100101001 |
| C₄ | 47 | 01111011110010101110010011011000101011000010000 |
| C₅ | 59 | 10100010101101100010000110000011111001111011100100101011101 |

The specific implementation of the encoded DNA data storage method with accompanying composite codes as index includes the following steps:
(1) The 5 selected component codes were each subjected to periodic extension to obtain the periodic extension sequence of each component code, all with a length of 129374315. Then, majority decision was adopted as the Boolean combination logic for constructing a composite code, and bitwise operations were performed to obtain a composite code with a period P=129374315, which was represented in binary form;
(2) The initial phase of the composite code was predefined. Then, a data sequence and the composite code were partitioned, with each group having a length of 130 bits, yielding 995187 shortened composite codes and data sequences, respectively. These were paired sequentially to form dual-layer bit sequences;
(3) According to the mapping rule {00→A, 01→T, 10→G, 11→C}, bit pairs were converted into bases, and 995187 DNA sequences with a length of L=130 were finally obtained;
(4) Random errors were added to each sequence to generate simulated sequencing reads, where the error rates of insertion (Ins.), deletion (Del.), and substitution (Sub.) satisfied P_{Sub.}=2P_{Ins}=2P_{Del.}=0.0050, and Monte Carlo repeated experiments were performed;
(5) The sequencing reads were directly demapped to obtain a corrupted composite code and a data bit sequence;
(6) The 5 known component codes were utilized to perform sliding correlation detection with the corrupted composite code, respectively, with a total of 31+35+43+47+59=215 component code correlation operations executed. The estimated phase of each component code correlation peak was searched, and then the unique index of the current read was derived by calculation through the Chinese Remainder Theorem;
(7) The known component codes and the identified estimated phase of each component code were utilized to regenerate an ideal composite code with a length of 130 bits, and in-phase autocorrelation operation was performed with the corrupted composite code. The normalized autocorrelation value was compared with a preset threshold, which was set to 0.8. If the autocorrelation value was greater than or equal to the preset threshold, the position index identification was valid; otherwise, the read was discarded. In the enhanced mode, reads with autocorrelation values lower than the preset threshold were retained. An edit distance-based alignment method was adopted to align the corrupted composite code with the known full-length composite code, and the alignment position corresponding to the minimum edit distance was located as the index. Insertion and deletion error patterns were identified and corrected according to the alignment path; and
(8) According to the identified data position information, consensus and decoding were performed on the corrupted data sequence to recover the original data.

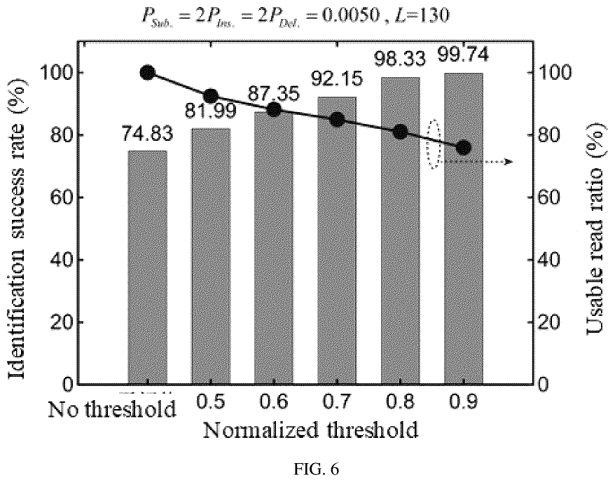

FIG. 6 presents a performance comparison between read identification accuracy and a usable read ratio under different thresholds. Simulation results show that when the number of component codes is 5 and the check threshold is 0.8, the read identification accuracy can reach over 98%, and the usable read ratio approaches 80%, verifying that the method proposed by the present invention can achieve highly robust identification in scenarios including insertion/deletion errors.

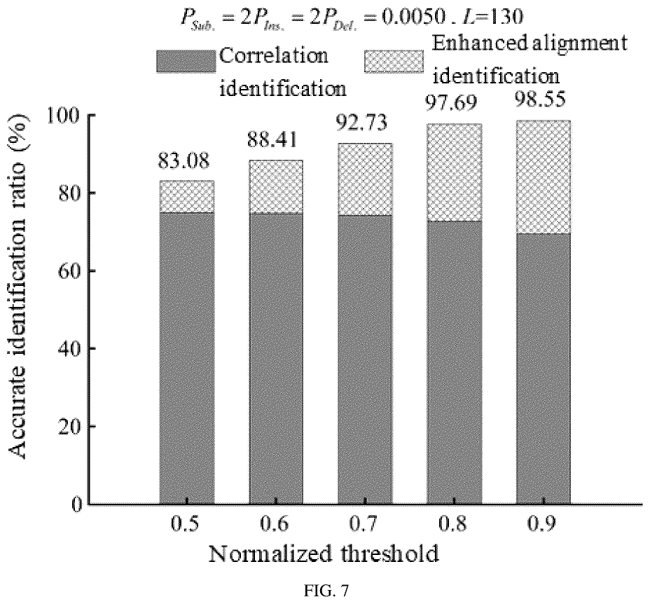

FIG. 7 presents a proportion of accurately identified reads in the enhanced mode. Simulation results indicate that when the threshold is set to 0.8 or higher, the proportion of correctly identified reads among all reads increases to 97.69%, indicating that the enhanced mode of the method proposed by the present invention can effectively increase the number of usable valid reads while ensuring the accuracy of read identification.

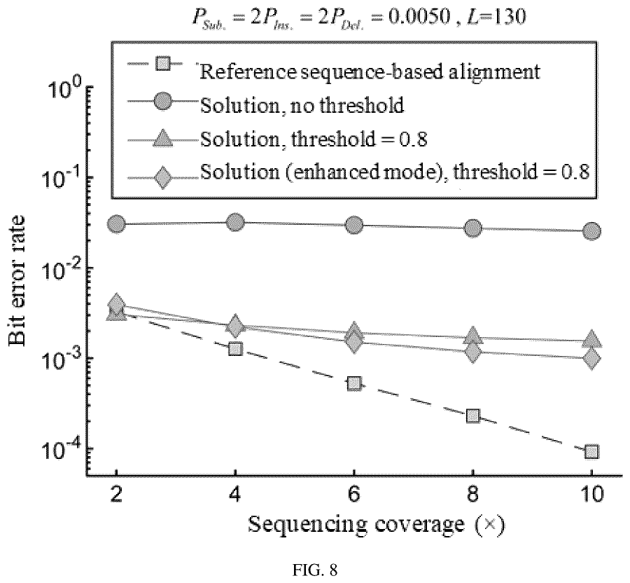

FIG. 8 presents a bit error rate performance of the method proposed by the present invention under different sequencing coverages. Simulation results show that setting the threshold check can significantly improve the data bit error rate after multi-copy consensus. When the enhanced mode is adopted, the error rate performance is further improved with the increase of the sequencing coverage, approaching that of the reference sequence alignment-based method.

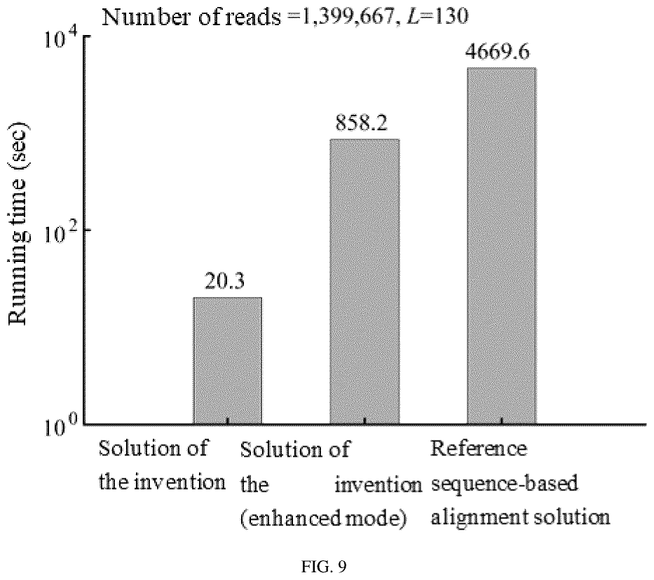

FIG. 9 presents a comparison of time complexity performance. 1399667 reads with a length of 130 nucleotides were identified and their running time was counted. The results show that the processing speed of the method proposed by the present invention is approximately 230 times faster than that of the reference sequence alignment-based method, significantly improving read identification efficiency. Furthermore, when the enhanced mode is adopted, the processing speed is about 5 times faster than that of the reference sequence alignment-based method, verifying the low implementation complexity of the method proposed by the present invention.

### Example 2

In this example, a composite code constructed from 3 component codes was adopted as the index of a data DNA. The identification performance of 130-nucleotide sequencing reads was simulated to further verify that the indexing method proposed by the present invention still has low complexity and high robustness when the number of component codes is 3. A component code set was formed by selecting 3 pseudo-random sequences with periods of 499, 503, and 511, respectively, where the component codes with periods of 499 and 503 are Legendre sequences, and the component code with a period of 511 is an m-sequence. Table 1 presents the bit sequence corresponding to each component code.

TABLE 2
3 pseudo-random component codes used to construct the composite code

| Component | Code | |
|---|---|---|
| code | length | Bit sequence |
| C₁ | 499 | 1011000110111101011100010001100010010100111010001010110 |
| | | 1001111110110100110011011000101110110111101110010000010 |
| | | 0011110100000000001100001100101100001111000110010010111 |
| | | 0001001010110010010010000011100000011110111101010100111 |
| | | 1011000001011000101011111010101010100000101011100101111 |
| | | 1001000011010101000010000111111000111110110110110010101 |
| | | 10111000101101100111000011110010110011110011111111110100 |
| | | 0011101111101100010000100100010111001001100110100100000 |
| | | 0110100101011101000110101101110011101110001010000100111 |
| | | 001 |
| C₂ | 503 | 01111011110111101010011111111000110010010011101111101010 |
| | | 1001010110110100110101110101111010011011111110001000100 |
| | | 0110001100110001011011010001001011111001000111010011000 |
| | | 1011101101110000111100111010101111110001011000000010010 |
| | | 0011110010101101100001110000100110111100011110010010101 |
| | | 1000011101101111111001011100000010101000110000111100010 |
| | | 0100010111001101000111011000001011011101001001011100110 |
| | | 0111001110111011100000001001101000010100010100110100100 |
| | | 101011010101000001000110110110011100000000110101000010 |
| | | 00010000 |
| C₃ | 511 | 1111111101111011100111101100011010101001111001000010110 |
| | | 0100011011101011110101001011000000100110110110100100000 |
| | | 0110110010101100110011111110011100110101110010110100000 |
| | | 0010111010011100010100110100110000111000001000101111100 |
| | | 1010010010001001111101001010000010101010111111010110101 |
| | | 0000110100010001111110001100010110110000101000101011101 |
| | | 1011110011000111101000010010011001011110001000011110000 |
| | | 0000011111000010000011101000110011011111011010110001001 |
| | | 0111000011000001100100111010101101110001110010010101000 |
| | | 1110110011101110 |

First, the 3 selected component codes were each subjected to periodic extension, and majority decision combination logic was adopted to construct a composite code with a period P=128259467, which was represented in binary form. Second, a data sequence and the composite code were partitioned, with each group having a length of 130 bits, yielding 986611 shortened composite codes and data sequences, respectively. These were paired sequentially to form dual-layer bit sequences, and after mapping, 986611 DNA fragments with a length of L=130 were obtained.

Further simulation was performed on the sequencing readout process. Random errors were added to each sequence to generate simulated sequencing reads, where the error rates of insertion (Ins.), deletion (Del.), and substitution (Sub.) satisfied P_{Sub.}=2P_{Ins.}=2P_{Del}=0.0050

The specific implementation of data reading refers to steps (5)-(8) in Example 1, with the difference that 3 known component codes were adopted for sliding correlation detection in this example.

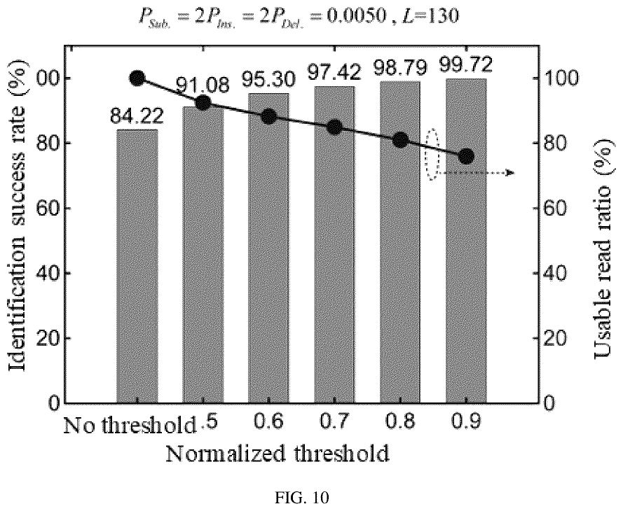

FIG. 10 presents a performance comparison between read identification accuracy and a usable read ratio under different thresholds. Simulation results show that when the number of component codes is 3 and the check threshold is 0.8, the read identification accuracy can reach over 98%, and the usable read ratio approaches 80%, verifying that the method proposed by the present invention can still achieve highly robust identification in scenarios including insertion/deletion errors when the number of component codes selected is 3.

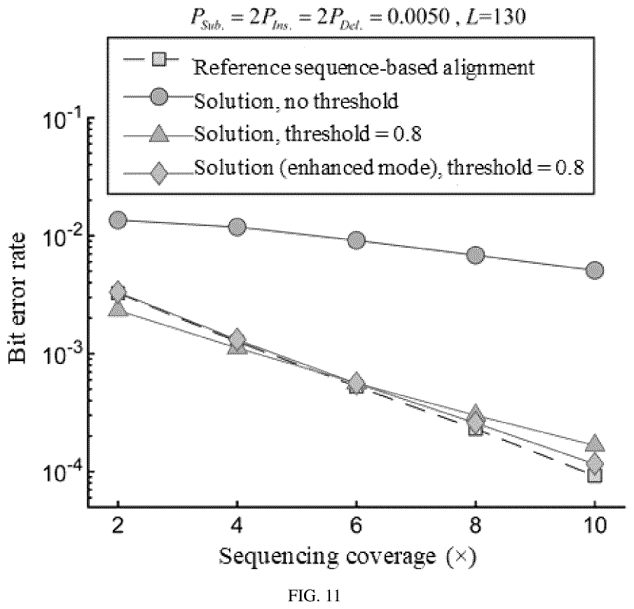

FIG. 11 presents a bit error rate performance of the method proposed by the present invention under different sequencing coverages. Simulation results show that after setting the threshold check, when the sequencing coverage is around 5×, the error rate performance of both the non-enhanced mode and the enhanced mode is basically consistent with that of the reference sequence alignment-based method, verifying that the proposed method can achieve performance comparable to the reference sequence alignment-based method when the number of component codes selected is 3.

### Example 3

This example demonstrates the readout and recovery of a large DNA fragment via next-generation sequencing. The component code set composed of 5 component codes in Example 1 was selected, and the block error correction code adopted a non-binary LDPC (22680, 7560) code with a code rate R=1/3

For the data storage with large DNA fragments, the specific implementation of encoding with accompanying composite codes is as follows:

The composite code construction process was the same as that in Example 1, resulting in a composite code with a period P=129374315. User data was encoded using the non-binary LDPC (22680, 7560) code to obtain a codeword with a length of 22680 bits, which formed a data sequence. The initial phase of the composite code was predefined and the full-length composite code was shortened to obtain a shortened composite code with a length of 22680 bits. This shortened composite code and the data sequence then formed a dual-layer bit sequence, which was transcoded according to a mapping rule between a bit pair and a base {00→A, 01→T, 10→G, 11→C}, ultimately yielding a large DNA fragment with a length of 22680 bases.

The next-generation genome sequencing data simulation software ART was utilized to simulate the random fragmentation and sequencing processes of the large DNA fragment. A single-end 150 sequencing mode was set, where the error rates of insertion, deletion, and substitution satisfied P_{Sub.}=2P_{Ins.}=2P_{Del.}=0.0050 thereby obtaining short sequencing reads with a length of 150 nucleotides. The specific operations for readout and recovery were consistent with steps (5)-(8) in Example 1.

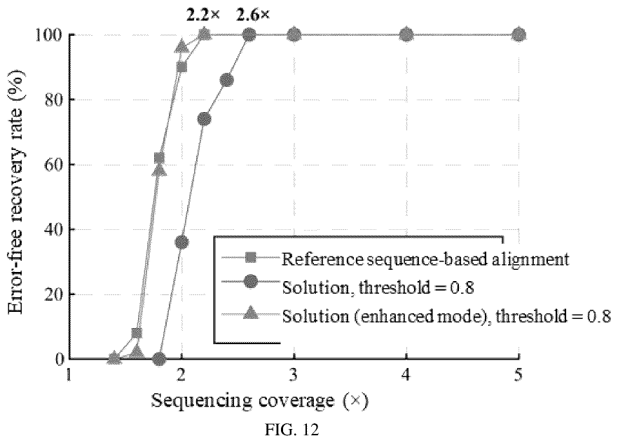

FIG. 12 presents the error-free recovery results from 50 sets of repeated experiments under different sequencing coverages. The results show that the method proposed by the present invention can achieve error-free recovery at a sequencing coverage of 2.6× (a well-known term in the art). After the enhanced mode was adopted, error-free recovery can be achieved at a sequencing coverage of 2.2×, which is comparable to the performance of the reference sequence alignment-based method. This demonstrates that the method proposed by the present invention can realize complete error-free recovery of original data at relatively low sequencing coverage.

### Example 4

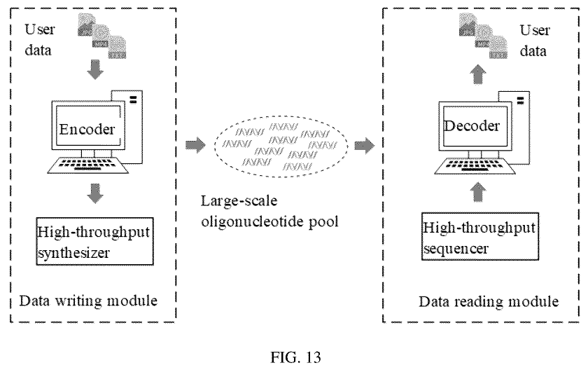

The storage method can be applied to a large-scale DNA data storage system based on encoding with accompanying composite codes, as shown in FIG. 13. The system includes a processor configured to execute the steps of the storage method. During data writing, the processor uses a composite code constructed from a limited number of pseudo-random component codes as an accompanying index, which is combined with encoded data bit sequence and transcoded to obtain a short DNA oligonucleotide. During data reading, the processor utilizes a limited number of component codes for finite-length sliding correlation acquisition with a corrupted composite code to achieve robust indexing of a short fragment, and performs consensus and decoding on corrupted data to achieve reliable data recovery.

The processor includes a data writing module and a data reading module.

The data writing module includes an encoder and a high-throughput synthesizer.

The working process of the encoder includes the following steps:
(1) Five pseudo-random sequences with periods of 4095, 4099, 4111, 4127, and 4139 were selected as component codes, where the component code with a period of 4095 adopts an m-sequence, and the component codes with periods of 4099, 4111, 4127, and 4139 adopt Legendre sequences;
(2) The 5 selected component codes were each subjected to periodic extension to obtain the periodic extension sequence of each component code. Then, majority decision was adopted as the Boolean combination logic for constructing a composite code, and bitwise operations were performed to obtain a composite code with a period P=1178716048165725615, which was represented in binary form;
(2) The initial phase of the composite code was predefined, then user data and the composite code were partitioned, with each group having a length of 255 bits, and sequentially paired to form dual-layer bit sequences, where the upper limit of the user data scale can reach approximately 130.86 PB, and the user data can also include redundant bits added by linear block error correction coding; and
(3) Bit pairs were converted into bases according to a preset mapping rule to construct 4622415875159 data DNA sequences with 255 nucleotides. 20-nucleotide amplification primers were added to both ends of each sequence, ultimately forming complete DNA sequences with 255+20+20=295 bases.

The high-throughput synthesizer takes the DNA sequences generated by the encoder as input and is configured for highly parallel synthesis of information-carrying DNAs to obtain a large-scale oligonucleotide pool.

The data reading module includes a high-throughput sequencer and a decoder.

The high-throughput sequencer is configured to perform highly parallel sequencing on a DNA library sample prepared by library preparation, and generate sequencing read data through base calling.

The decoder takes the sequencing data generated by the high-throughput sequencer as input, and its working process includes the following steps:
(1) The sequencing reads were directly demapped to obtain a corrupted composite code and a data bit sequence;
(2) The 5 known component codes were utilized to perform sliding correlation detection with the corrupted composite code, respectively, with a total of 4095+4099+4111+4127+4139=20571 component code correlation operations executed. The estimated phase of each component code correlation peak was searched, and then the unique index of the current read was derived by calculation through the Chinese Remainder Theorem;
(3) The known component codes and the identified estimated phase of each component code were utilized to regenerate an ideal composite code with a length of 255 bits, and in-phase autocorrelation operation was performed with the corrupted composite code. The normalized autocorrelation value was compared with a preset threshold, which was set to 0.8. If the autocorrelation value was greater than or equal to the preset threshold, the index identification was valid; otherwise, the read was discarded. In the enhanced mode, reads with autocorrelation values lower than the preset threshold were retained. An edit distance-based alignment method was adopted to align the corrupted composite code with the known full-length composite code, and the alignment position corresponding to the minimum edit distance was located as the index. Insertion and deletion error patterns were identified and corrected according to the alignment path; and
(4) According to the identified data position information, consensus and decoding were performed on the corrupted data sequence to recover the original data.

It can be understood by those skilled in the art that the drawings are merely schematic diagrams of a preferred example, and the above serial numbers of the examples of the present invention are only for descriptive purposes and do not indicate the merits of the examples.

It should be understood by those skilled in the art that the examples of this application may be provided as methods, systems, or computer program products. Therefore, this application may take the form of an entirely hardware example, an entirely software example, or an example combining software and hardware aspects. Furthermore, this application may take the form of a computer program product implemented on one or more computer-usable storage media (including but not limited to disk storages, CD-ROMs, optical storages, etc.) having a computer-usable program code included therein. The solutions in the examples of this application may be implemented using various computer languages, such as object-oriented programming languages, e.g., C++ and Python, etc.

This application is described with reference to flow charts and/or block diagrams of methods, devices (systems), and computer program products according to the examples of this application. It should be understood that each process and/or block in the flow charts and/or block diagrams, and combinations of processes and/or blocks in the flow charts and/or block diagrams, may be implemented by computer program instructions. These computer program instructions may be provided to a processor of a general-purpose computer, a special-purpose computer, an embedded processor, or other programmable data processing devices to produce a machine, such that the instructions executed by the processor of the computer or other programmable data processing devices generate means for implementing the functions specified in one or more processes of the flow charts and/or one or more blocks of the block diagrams.

These computer program instructions may also be stored in a computer-readable memory that can guide a computer or other programmable data processing devices to work in a specific manner, such that the instructions stored in the computer-readable memory generate an article of manufacture including instruction means, which implement the functions specified in one or more processes of the flow charts and/or one or more blocks of the block diagrams.

These computer program instructions may also be loaded onto a computer or other programmable data processing devices, such that a series of operational steps are executed on the computer or other programmable devices to generate computer-implemented processing, thereby the instructions executed on the computer or other programmable devices provide steps for implementing the functions specified in one or more processes of the flow charts and/or one or more blocks of the block diagrams.

The above descriptions are only preferred examples of the present invention and are not intended to limit the present invention. Any modifications, equivalent substitutions, improvements, etc. made within the spirit and principles of the present invention shall be included in the scope of protection of the present invention.

What is claimed is:

1. An encoded DNA data storage method with accompanying composite codes as index, comprising:
(1) at a data writing end, selecting n pseudo-random sequences as component codes, where n is a positive integer, and constructing a composite code as an index through combination logic, encoding user data using error correction codes to obtain an encoded data bit sequence, then performing accompanying encoding with bitwise combination on the composite code and the data bit sequence to form a dual-layer bit sequence, and transcoding it to obtain a data DNA sequence according to a mapping rule between a bit pair and a base, and finally completing data writing through artificial DNA synthesis; and
(2) at a data reading end, obtaining sequencing reads of a short-fragment DNA using next-generation high-throughput sequencing technology, preprocessing the sequencing reads, performing demapping according to the same mapping rule as the writing end, extracting a corrupted composite code and a corrupted data sequence, next, performing sliding correlation detection with the corrupted composite code using n known component codes respectively to obtain a correlation peak phase estimate for each component code, then performing calculation through the Chinese Remainder Theorem to determine unique indices of reads, then calculating an in-phase autocorrelation value of the composite code and performing threshold check, retaining reads that pass the check as they are while re-identifying reads that fail the check using an enhanced mode, and locating and correcting an insertion/deletion error in the corrupted data sequence, and finally, according to identified indices, using a plurality of copies of a sequence at the same position, performing a bitwise majority voting to generate a consensus data sequence, and sending it to a decoder for decoding to recover original user data.

2. The encoded DNA data storage method with accompanying composite codes as index according to claim 1, wherein the step (1) comprising:
(1.1) selecting n pseudo-random sequences as component codes to form a component code set {C₁, C₂, . . . , Cₙ}, then subjecting each component code to periodic extension to obtain sequences each with a length of P=p₁p₂ . . . pₙ, and performing bitwise calculation according to preset Boolean combination logic to construct a composite code a with a length of P=p₁p₂ . . . pₙ, and representing the composite code in binary form; where C denotes a component code n with a code length of pₙ, and n≥1 and is an integer;
(1.2) performing block error correction encoding on user data using m linear block codes (N,K) to obtain an encoded data bit sequence b; where m is the number of codewords, m≥1 and is an integer, N is a length of an encoded codeword, and K is a length of information bits; and since a period of the composite code can be designed to be extremely long, a single composite code can carry a plurality of block codewords, satisfying mN≤p₁p₂ . . . pₙ; and
(1.3) performing accompanying encoding with bitwise combination on the composite code a and the data bit sequence b to construct a dual-layer bit sequence, transcoding it into a DNA sequence according to a mapping rule between a bit pair and a base {00→A, 01→T, 10→G, 11→C}, and obtaining a long DNA fragment or a short oligonucleotide molecule by means of artificial synthesis technology;
where the preset Boolean combination logic specifically includes: logical multiplication, modulo-2 addition, and majority decision;
where the n pseudo-random sequences with short periods are selected as the component codes, satisfying: periods of the selected pseudo-random sequences are strictly coprime to each other, and each has sharp periodic autocorrelation characteristics; and
where the step (1.3) is specifically as follows:
for data storage with large fragments, first intercepting the composite code to obtain a shortened composite code with the same length as the data bit sequence, then aligning the shortened composite code and the data bit sequence end-to-end to form a dual-layer bit sequence, and directly performing mapping to obtain long DNA fragments; and for data storage with short oligonucleotides, first dividing the data bit sequence evenly into mN/l groups according to a length l, simultaneously performing sliding interception on the composite code according to a step size 1 to obtain mN/l shortened composite codes, then pairing the shortened composite codes with a length l and the data bit sequence in order to construct mN/l groups of dual-layer bit sequences, and finally, generating mN/l short-fragment oligonucleotides after mapping; where l is a positive integer.

3. The encoded DNA data storage method with accompanying composite codes as index according to claim 1, wherein the step (2) comprising:
(2.1) for a short-fragment oligonucleotide molecule pool, after direct library preparation, reading through next-generation high-throughput sequencing technology to obtain sequencing reads of a data DNA; and for a long-fragment data DNA, performing sequencing readout using high-throughput shotgun sequencing, and specifically, randomly fragmenting enriched DNA molecules, and reading through next-generation high-throughput sequencing to obtain sequencing reads;
(2.2) preprocessing the sequencing reads, performing demapping according to the same mapping rules as the writing end, and separately extracting a corrupted composite code â and a corrupted data bit sequence \hat{b} from a dual-layer structure; specifically, for sequencing reads of a short-fragment oligonucleotide, trimming off primer regions at both ends by aligning with amplification primers, and performing demapping on a retained payload; and for reads obtained by shotgun sequencing of a long-fragment DNA, performing demapping directly on the sequencing reads;
(2.3) sending the corrupted composite code a into a cross-correlation detector of each component code, performing sliding correlation acquisition using a known component code set {C₁, C₂, . . . , Cₙ} searching for a position of a correlation peak of each component code (i.e., a phase corresponding to in-phase correlation), obtaining an estimated phase of each component code, and then calculating through the Chinese Remainder Theorem to determine unique indices of reads;
(2.4) regenerate an ideal composite code a′ with the same length and phase as the corrupted composite code using the above obtained estimated phase of each component code, calculating the autocorrelation value of the composite code, and if an autocorrelation value meets a preset condition, retaining reads; and for reads that fail a check, performing re-identification using an edit distance alignment-based method in an enhanced mode to determine indices, and retaining the reads, while identifying and correcting insertion and deletion error patterns according to an alignment path; and
(2.5) considering that a plurality of copies of a data sequence may exist at the same position, performing a bitwise majority voting decision on the data sequence to obtain a consensus data sequence, and if no uniqueness exists in a voting decision, filling a bit at the position using the same erasure marker as above to obtain a codeword, and then sending it to a decoder to correct residual errors and recovering original user data.

4. The encoded DNA data storage method with accompanying composite codes as index according to claim 3, wherein the step (2.3) is specifically as follows:
(3.1) subjecting each component code in a known component code set to periodic extension, subjecting each component code to sliding correlation operation with the corrupted composite code, where a correlation window length is the same as a length of the corrupted composite code, a sliding number is a component code period, and a cross-correlation function between a component code i and the corrupted composite code can be expressed as:
$${R}_{\overline{r} , {\overline{C}}_{i}} ( τ ) = {\sum }_{j = 0}^{L - 1} \overline{r} ( j ) {\overline{C}}_{i} ( j - τ ) ,$$
where C̅ᵢ is a periodic extension sequence of the component code i, r̅ is the corrupted composite code, τ is a component code phase offset, L is the correlation window length, 1≤i≥n, the periodic extension sequence C̅ᵢ and the composite code r̅ are represented by symbols {+1, −1}, which satisfy a mapping relationship {+1→0, −1→1} with a binary sequence;
(3.2) according to a sliding correlation value between each component code and the corrupted composite code, searching for a position of a correlation peak to obtain a phase estimate of each component code, where a phase τᵢ corresponding to the correlation peak of the component code i can be expressed as:
$${τ}_{i} = {arg max}_{τ} ( \vert {R}_{\overline{r} , {\overline{C}}_{i}} ( τ ) \vert ) ,$$
where argmax represents obtaining the position corresponding to the correlation peak; and
(3.3) using an estimated phase of each component code obtained from the searching, calculating through the Chinese Remainder Theorem to obtain a unique index of a corresponding read, where the estimated phase of each component code satisfies
$$\{ \begin{matrix}φ ≡ {τ}_{1} ( mod {p}_{1} ) \\ φ ≡ {τ}_{2} ( mod {p}_{2} ) \\ ⋮ \\ φ ≡ {τ}_{n} ( mod {p}_{n} )\end{matrix} ;$$
where φ is the unique index of the read obtained through calculation, and τ₁, τ₂, . . . , and τₙ correspond to estimated phases of n component codes, respectively.

5. The encoded DNA data storage method with accompanying composite codes as index according to claim 3, wherein the step (2.4) is specifically as follows:
(4.1) according to the estimated phase of each component code mentioned above, regenerating an ideal composite code a′ with the same phase and length as the corrupted composite code â, and then calculating an in-phase correlation value between the corrupted composite code â and the ideal composite code a′, where the obtained correlation value can be expressed as:
$${R}_{r , {r}^{′}} = {\sum }_{j = 0}^{L - 1} r ( j ) {r}^{′} ( j ) ;$$
where r and r′ are the corrupted composite code and the ideal composite code, respectively, both represented by symbols {+1, −1}, and satisfy a mapping relationship {+1→0, −1→1} with the binary sequence;
(4.2) if the in-phase autocorrelation value of the composite code satisfies |R_{r,r′}|≥ωL, passing a check, and retaining a current read, where ω represents a preset correlation threshold, and 0≤ω≤1;
(4.3) for reads that fail the above autocorrelation check, in an enhanced mode, adopting an edit distance alignment-based method to compare the corrupted composite code â with the known complete composite code a, searching for an alignment position with a minimum edit distance as an index of a read, while locating positions with insertion and deletion errors in the corrupted composite code using an alignment path, and according to a characteristic that positions with insertion and deletion errors in a dual-layer sequence structure are completely identical, correcting insertion and deletion error patterns in the corrupted data sequence; discarding bits at positions with insertion errors, marking bits at positions with deletion errors as erasures and filling them with a predefined marker; and if the enhanced mode is not adopted, skipping this step.
